(19) United States
(12) Patent Application Publication
(10) Pub. No.: US 2020/0129811 A1
(43) Pub. Date: Apr. 30, 2020

(54) Method of Coaching an Athlete Using Wearable Body Monitors

(71) Applicant: Benjamin Douglas Kruger, Lexington, KY (US)

(72) Inventor: Benjamin Douglas Kruger, Lexington, KY (US)

(21) Appl. No.: 16/732,574

(22) Filed: Jan. 2, 2020

Related U.S. Application Data
(63) Continuation-in-part of application No. 15/473,125, filed on Mar. 29, 2017, now Pat. No. 10,548,511.

(51) Int. Cl. A63B 24/00; A63B 71/06; A61B 5/11

(52) U.S. Cl. CPC A63B 24/0075; A63B 71/0622; A61B 5/11; A63B 2024/0068; A63B 2024/0081; A63B 2220/803; A63B 2220/836; A63B 2225/20

(57) ABSTRACT

A method of providing coaching or teaching a training regimen, trick shot, athletic motion, workout program, ergonomic trade secret. The method of providing coaching information by a user at a remote location to another user at another remote location via digital communication. The coaching information is obtained from sensors worn on the body of an athlete. The sensors provide both biomechanics and physiological data. The sensors provide data to a server that can analyze the movement of the athlete. The analyze data can be stored and labeled. The data can then be accessed for display on another users preferred device. The user can then attempt the motion and will receive biofeedback from the sensors worn.

### CROSS REFERENCE TO RELATED APPLICATIONS

This application is a continuation in part of and claims priority to U.S. patent application Ser. No. 15/473,125, filed on Mar. 29, 2017, and incorporated fully herein by reference.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The invention relates to a system and method of using body monitors on an user for analyzing and archiving a coaching, training regimen, trick shot, athletic motion, workout program, ergonomic trade secret, or any other motion a user wants to teach another user for the purpose of displaying it on another user's preferred device in another remote location.

### BACKGROUND OF THE INVENTION

The invention relates to methods and apparatus for sports training. In particular, coaching, analysis and feedback system is provided for remote users. Through collecting data from sensors attached to garment worn by a user, or original user, a coaching lesson may taught to another user, or end user, at a different remote location.

Users use a variety of techniques to improve their performance. Practice, sport specific strength training, and sport specific diets are all very common. Throughout history, coaches have attempted to determine the best way to perform a specific athletic endeavor. Many coaches prefer a hands-on approach when analyzing a user's athletic performance. Many coaches use the so called “eye test”, meaning they watch the motion, and provide feedback to the user. Coaches based their feedback on prior history and knowledge of watching different approaches that worked for different users. This method turns into trial and error and a lot of frustrating moments throughout the process. One disadvantage to this method is the amount of time it takes a human being to blink and the amount of time it takes an experienced user to complete a motion like a swing. These two can overlap and cause problems in the coaching process. Another disadvantage is coaches are unable able to accurately decipher the current physiological scenario of the user, including the amount of muscle fiber activation rates, amount of lactic acid, amount of fatigue, the current heart rate, and other variables which significantly affect the particular motion, and in turn the overall performance of a user. The last disadvantage of the eye test is the instructions can be “lost in translation”. By attempting to show the user how to correctly preform the motion, the coaches motion will not be an exact replica of what the coach thinks the motion should be. There will be deviations in the process, which leads to confusion.

Other coaches use video cameras to record the user and show the user what they did wrong. The major disadvantage for this process is, even though we can repeatedly study the user's motion, the internal body features are unknown. We are still using the “eye test” based on other users' performances to better the user's motion.

Modern technology has vastly improved the ability to train a user to perfect the various aspects of his or her sport. It is common to video record users and allow them to see themselves in action. This allows the user and a coach to evaluate every aspect of the user's performance, from their fundamentals to their game related behavior. A basketball player can watch game tapes to see how they were shooting, and to evaluate, in slow motion, what they did wrong during a particular shot.

There are now numerous sensors that can be used to assist users. The most common and well known is the FitBit® which measures the number of steps a person takes, but can also measure heart rate, blood pressure and body temperature. All of that data can help a user and coach evaluate the user's performance. Another common technique is to apply video sensitive tape (or tight-fitting clothing with video sensitive reflectors) and video the user during simulated aspects of their sport-a golf swing, a pitcher throwing-and develop a computer model of the specific user's body movement. This can be done for every aspect of the particular user's sport. This allows the coach to evaluate the specific body movements for efficiency and maximum performance. Runners, for example, can determine the most effective leg movement to increase speed or endurance.

The exponential growth in technology provides new ways to analyze athletic performance. There are a number of different sensors that can be attached directly to the user's body, or equipment, to provide data on the user's movement during a specific athletic event, such as swinging a golf club. These sensors can be attached directly to the skin by use of tape. These sensors can also be attached to the clothing that the user wears. The three most common types of sensors are the inertial monitoring unit, the IMU, and the surface electromyography, or SEMG, monitors, and electrocardiogram, or EKG, monitors. The SEMG monitors muscle fibers, through surface electromyography SEMG. The EKG's main function is to monitor the said user's heart rate. The IMU monitors the said user's motion.

These sensors can provide a good deal of information about an user's body motion while performing an athletic task, like swinging a golf club, throwing a pitch, shooting a basket, and the like. This information can be used to help the user improve performance. It would be valuable to further analyze this data to help the user improve performance by incorporating strength training and diet.

### SUMMARY OF THE INVENTION

The invention relates to a system and method of using body monitors on an user, or original user, for analyzing and archiving a coaching, training regimen, trick shot, athletic motion, workout program, ergonomic trade secret, or any other motion an original user wants to teach another user, or end user, for the purpose of displaying it on the end user's preferred device in another remote location.

The sensors capture and analyze the body movements of the user, as well as the specific physiological conditions of the user's body, such as heart rate, muscle activation, and other electromagnetic activity results within the user's muscles and heart. The invention's sensors are contained in skin tight clothing worn by the user. The sensors transmit information to a separate preferred method, more specifically a computer, tablet, or cellular device. From which, the data is sent to a server which contains software that records, analyzes, and archives the data. Such archived data may be displayed on another users, or end users, preferred device.

The original user may edit any aspect of a coaching, training regimen, trick shot, athletic motion, workout program, ergonomic trade secret, or any other motion an original user wants to teach another user (may be referred simply as training regimen throughout the patent) after it is archived. The original user may also edit any aspect of the training regimen based on the end user's physiological values, or anything such, recorded from the sensors.

The biofeedback provides information to the user to allow them to modify their movements and obtain a more correlated motion between the original users archive motion and the real time motion completed by the end user.

### DESCRIPTION OF THE DRAWINGS

FIG. 1 is a flow chart of a method for using data collected from sensors for the purpose of analyzing the data, and archiving the data (FIG1coaching)

FIG. 2 is a flow chart of a method for biofeedback (FIG2coaching)

FIG. 3 is an information flow diagram for the apparatus of the invention (FIG3coaching)

FIG. 4 shows the location of the IMU, and the electrodes on the front of the compression shirt

FIG. 5 shows the location of the IMU, and the electrodes on the back of the compression shirt

FIG. 6 shows the location of the IMU, and the electrodes on the front of the compression pants

FIG. 7 shows the location of the IMU, and the electrodes on the back of the compression pants

FIG. 8 shows the location of the IMU, and the electrodes on the front of the compression shorts

FIG. 9 shows the location of the IMU, and the electrodes on the back of the compression shorts

### DETAILED DESCRIPTION OF THE INVENTION

The invention uses data 300 collected through sensors 200 to calculate the specific motions for the purpose of coaching or teaching a training regimen, trick shot, athletic motion, workout program, ergonomic trade secret, or any other motion a user wants to teach another user for the purpose of displaying it on another user's preferred device in another remote location. These sensors 200 include electrodes (or surface electromyography 201, also referred by SEMG sensors and Electrocardiogram 201, also referred to by EKG or ECG) and Inertial Measurement Units, also referred by IMU sensors 202, which are a combination of two or more of accelerometers, gyroscopes, magnetometers, and barometers. These sensors 200 are embedded in or are attachable to clothing 100 worn on the user's body. The data 300 is collected and sent wirelessly from a microcontroller 203, which is attached to the clothing 100, to a preferred method 204, either a cellular device, tablet, or computer. The IMU sensors 202 may be attached and powered through microcontroller 203, or MCU, or the IMU sensors 202 may be attached and powered through something similar in nature. The data 300 is then sent to a server 205 to be run through algorithms. The coaching or teaching of a training regimen, trick shot, athletic motion, workout program, ergonomic trade secret, or any other motion a user, or original user, wants to teach another user, or end user, is available through digital communication for any end user in any remote location for download. The information provided after the download is completed is displayed and biofeedback after every attempt may be relayed to the said end user.

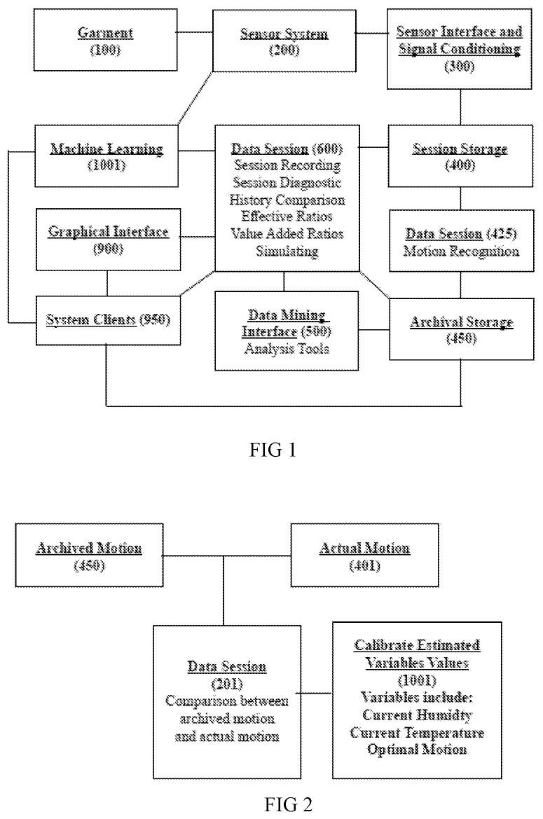

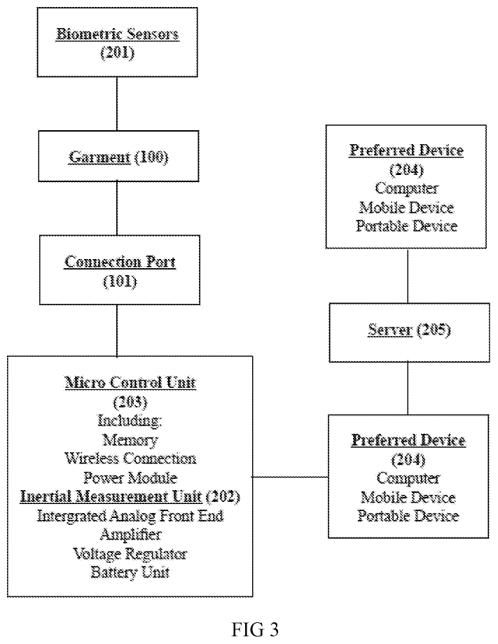

FIG. 1 is a flow chart of a method for using data collected from sensors for the purpose recognizing a motion, analyzing a motion, and archiving a motion. FIG. 2, FIG. 3, show more details of specific processes included in FIG. 1. FIG. 2 is a flow chart of a method for biofeedback. FIG. 3 is an information flow diagram for the apparatus of the invention. This shows the connection between the sensors and the order the data is relayed.

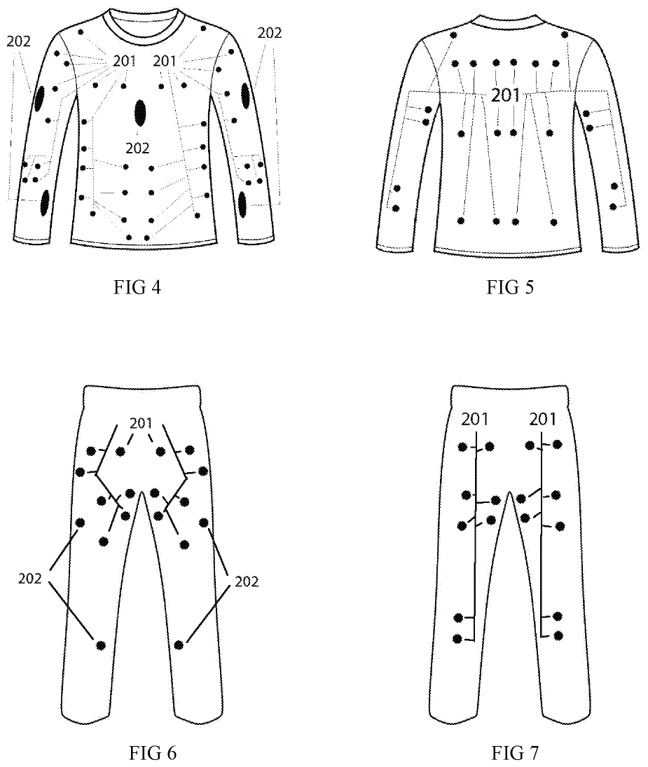

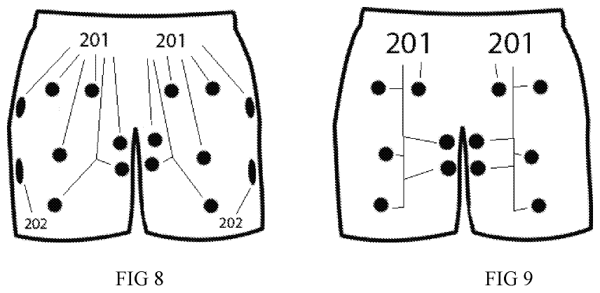

FIG. 4 and FIG. 5 show the location of the 200 sensors in the shirt 100. The sensors 201 are electrodes used in the Surface Electromyography, or SEMG, and Electrocardiography, or EKG. The sensors 202 are attachable microcontrollers that consist of at least an inertial measurement unit, or IMU, memory, Wireless Connection, Power Module, Integrated Analog Front End, Amplifier, and a Voltage Regulator. FIG. 6 and FIG. 7 show the location of the sensors 200 in the compression pants 100. FIG. 8 and FIG. 9 show the location of the sensors 200 in the compression shorts 100. There are two articles of clothing 100, a shirt and compression pants, or compression shorts. The clothing 100 is skin tight to allow the sensors 200 to be directly against the user's skin. The sensors 200 may be incorporated into the clothing by sewing or other appropriate methods.

Processors may be one or more conventional processor, like a CPU or GPU, or other including, but not limited to, an ASIC, FPGA, or other hardware based processors. Processors may or may not work in parallel with other processors. Processors may execute any code, or instructions. This includes portions of instructions in some cases. For example, a processor may only execute a portion of a set of instructions to save time and processing power.

The executable code may be stored in an external memory, on a processor, or any other such. Executable code may give instructions directly or indirectly from the processor. These instructions, or executable code (which are used interchangeably throughout), may be loaded onto any processor, external storage, and/or other through, but not limited to, a USB Cable, Bluetooth, Bluetooth Low Energy (or BLE), RFID, WiFi, or NFC. The instructions may be stored in object-code format or any other computer language.

Instructions from the processors, or such, may or may not change the sampling rate for any reason. Some reasons include, but are not limited to, noise reduction, power management, or any other such reason. For example, if noise artifacts are highly present, a set of instructions may or may not change the sampling rate to receive less values and more accurate results.

The memory stores any information including, but not limited to, algorithms, instructions, data, or other. The memory can be defined as anything capable of storing information and having that information retrieved from a processor. The memory can store information from one or more processors. The memory protects against overwriting, clocking, corrupting, interrupting, or any interference between any application, the sensor system, application processor, or any other components.

Data storage, archive storage, or a database (all of which are interchangeable) may store information, data, analytics, algorithms, or any other type the archive storage can read. The data can be formatted in any computing device readable format. The data may be retrieved, stored, or modified by instructions from a process or such.

Communication between any application, the sensor system 200, the application processor, or any components may be wired or wireless. Wireless communication includes, but not limited to, Bluetooth, Bluetooth Low Energy (BLE), WiFi, ANT, WLAN, Powerline networking, and cell phone 205 networks. Communication may or may not be bi-directional. Communication may transmit, communicate, push data to other devices, receive, request, pull data, and store data. Communication may act as a relay between devices and/or the internet. Communication may also include NFC, RFID, and other such. The sensors and other inputs may communicate with local and/or remote processing and storage devices via any suitable communications protocol and network.

The server, or network, may be managed by one or more networked backend servers and may include different databases for information on the user or the community. Information may include user information, performance information, content information, or anything similar.

Location can be calculated from a Global Positioning Device (or GPS), IMUs 202, laser based locational systems, LIDAR, camera-based localization systems, or anything related. The location can also be calculated by sensor fusion to increase accuracy. The sensor fusion can use algorithms like a Kalman filter, a convolutional neural network, a Bayesian Network, Central limit theory, or any other algorithm used for the purpose of uncertainty reduction. An example of information calculated from a locational based method is the longitude, latitude, and/or altitude position.

The data signal processing (or DSP) circuitry may include, but is not limited to, a MCU. The processor may or may not include or instruct a particle filter, a Kalman filter, a convolutional neural network, a Bayesian Network, Central limit theory, or any other algorithm used for the purpose of uncertainty reduction. The DSP can be implemented via instructions from a hardware, firmware, software, or any combination of the three.

The data may be displayed visually through a digital display. Such display include, but are not limited to, LED, LCD, Preferred Personal Device, Flexible display attached to the clothing/garment 100, and other display technologies. Any information, including data, may be displayed on a screen connected with the processors. These may be wireless or wired in to transmit data. This data can then be visually displayed, but in some cases the data will be conveyed through audio feedback. The audio feedback can be convey on any speaker connected or accessible to the processors.

The location of each Inertial Measurement Unit, or IMU 202 (used interchangeably), is also essential to this invention. FIG. 4 and FIG. 5 shows the locations of the sensors 200 on the front and back of the shirt 100, while FIG. 6, FIG. 7, FIG. 8, and FIG. 9 shows the locations of the sensors 200 on the front and back of the pants or shorts 100. These locations were picked because they can decipher 600 the exact motion 300 for the body at any given time. The IMU 202 consists of, but not limited to, an accelerometer, gyroscope, and a magnetometer. The data and states of the IMU 202 can be obtained and stored. The accelerometer can measure the acceleration around a 3D axis. The gyroscope can measure the angular velocity of the unit. And the magnetometer is used to measure the Earth's local magnetic field. By placing the IMUs 202 on a specific location on the body, the motion 300 of the body can be measured.

The states of the IMU 202 are then given instruction from the processor to calculate the angles of X, Y, Z. The process can use a variety models including using quaternions models. These models convert the raw states of the IMU 202 to analyzed data, which is in turn used for metrics and kinematics. Some metrics include, but is not limited to, the linear acceleration of a body segment and the angular velocity of a body segment.

Before the measurements are accurate, a calibration process needs to occur. In general practice in the industry, there have been three widely excepted IMU 202 location calibration procedures. These procedures include the static pose, functional calibration, and technical calibration. The static pose calibration requires the user to take a unique stationary pose. The functional calibration requires the user to complete a motion 300 around an imaginary axes. The technical calibration requires manually aligning the IMUs 202 with the bone structure. There are also other calibration procedures, not as widely accepted, but have been proven to be affective.

The sensor fusion algorithm assumes that the human body parts, or segments (used interchangeably), are rigid bodies. By making this assumption, a kinematic chain can be formed. The kinematic chain uses joint constraints to make the sensor fusion more accurate and modelled more like a human motion. Kinematic chains is more accurate by helping to prevent the drift of body segments over time. Although the kinematic chains are favored, the scope should not exclude free segment models.

The kinematic chain joint constraints are calculated at the beginning of using the invention the first time. The invention requires a set of motions 300 to be completed to decipher variations in natural flexibility in each direction for each specific joint. Similar to completing static poses to calibrate the IMUs 202, the invention requires a series of stretches and movements to calibrate the joint constraints. By solving for the joint constraints and completing correct kinematics using joint relation, unbounded integration drift is prevented. The kinematic chain also includes position and rotation constraints. The position and rotation constraints limit the drift due to the clothing 100 moving during the motion 300 or soft tissue artifacts.

The kinematic chain integrates all sensors 200 and their metric 600 including, but not limited to, muscle torque and acceleration. By considering integrated acceleration data, tracking for different movements and scenarios, like jumping and climbing a hill, can be calculated. By considering SEMG 201 values and metrics, a realistic movement can be calculated and a reduction in drift is achieved. The sensors 200 fusion allows the IMU 202 data to be cross referenced with the expected movement and its metrics when considering the muscle fiber activation. More specifically, a movement and its metrics 600, including velocity, should render a muscle fiber activation rate value in a combination of physiological cross sectional areas (or PCSA).

Some key body segments, including fingers, hand, feet, and head may or may not be monitored by sensors 200. In the scenario that a key body segment is not monitored by sensors 200, the key body segment's movement is estimated and included in the motion 300 database. Through algorithms similar to gait, or motion (used interchangeably), recognition, the algorithm is trained to associate key muscle fiber activation rates and movements with a simulated key body segment movement. The training data is collected through a series of motions 300 that are instructed to be completed through the display 900. That data allows the processors to look for key indicators of monitored body segments that correspond with the simulated body segments motion. The simulated gait recognition may use models including, but not limited to, generative adversarial networks (or GAN), Convolutional Neural Network (or CNN), Long-Short-Term memory (or LSTM), generalized recurrent units (or GRU), artificial neural network (or ANN), Bayesian Probability, genetic algorithms or any combination or such.

For example, the hand position is calculated by the combination of the IMUs 202 and the muscle fiber activation rate in key areas including the forearm. The forearm SEMGs 201 uses the motion recognition algorithm 425 to decipher the location and path of the fingers and wrist. These muscles are called extrinsic hand muscles, or more specifically include, but are not limited to, the Flexor Carpi Ulnaris, Palmaris Longus, Flexor Carpi Radialis, Pronator Teres, Flexor Digitorum Superficialis, Flexor Digitorum Profundus, Flexor Pollicis Longus, Pronator quadratus, Aconeus, Brachioradialis, Extensor Carpi Radialis Longus and Brevis, Extensor Digitorum, Extensor Digiti Minimi, and Extensor Carpi Ulnaris. The abduction, adduction, extension, and flexion movements of the muscles are used to calculate the location of the wrist and fingers through the motion recognition algorithm 425. The training data for the individual is a simple test, which is occurs usually at the beginning of collected data, or first time the garment 100 is being worn and data is collected through the sensors 200. For the hand example, the user, original user and/or end user, wearing the garment will be asked to complete a series of finger and wrist motion 300 which will be used to decipher the motions 300.

The location of the SEMG 201 sensors 200 allows the process to monitor the muscle fiber activation rate 300 for a select group of muscles. This is measured by the electromagnetic activity 600 in the muscles. The higher the activation rate, the more muscle contraction is evident. The invention also monitors for the increase in the activation rate of muscle fibers that are not used to their full potential 600, which is different from muscle hypertrophy. Muscle hypertrophy by definition is the increase in size of skeletal muscle through a growth in size of its component cells. The patent will coin the term muscle hypertrophy for both scenarios, even though they have different meanings. This invention monitors and analyzes the changes in the activation rate of muscle fibers in the monitored muscles.

By monitoring the changes in muscle fiber activation rate, this invention also accurately calculates 600 the fatigue of each monitored muscle, amount of possible force per muscle, which muscles are stronger than others, which muscles are used most often during an ergonomic or athletic motion 300, and which muscles are more susceptible for muscle hypertrophy. A clear sign of fatigue is the decrease in activation rate for fast twitch muscle fibers. These muscle fibers produce a signal of 126-250 Hz. The amount of force per muscle is calculated 600 by dividing the Newton's force by the cross-sectional area of the muscle. The amount force is than compared to all other muscles to decipher which muscles are stronger than the others. During a motion 300, the force divided by time t allows the process to decipher which muscles are most active. To tell the effects of fatigue for that particular user for the particular motion 300, the invention calculates the deviations of the motion 600, using the IMU 202 data against the amount of fatigue.

While lifting weights or performing an athletic motion 300, the activation rate of muscle fibers can be a clear sign of many key analytics 600 including, but not limited to, the maximum amount of force, the maximum amount of force velocity, and the time until fatigue occurs for the specific muscle. Since muscle fibers are responsible for movement, the velocity and the force of the motion 300 directly relate to the increase in muscle fibers. By using historical data 450, the process can estimate the amount of changes in force, velocity, and flexibility during fatigue or if muscle hypertrophy occurred. The process uses a regression equation to estimate the added amount of max velocity with respect to the fast twitch and slow twitch muscle fibers. For example, the regression equations stipulates that the user, on average, will increase velocity by X and force by Y for each increase in muscle fibers. During a motion 300, the percent of activation for each muscle 600 is a clear indicator which muscles are essential to the completion of the motion 300. If a lower than normal activation rate is noted, it can mean that there is wasted potential energy, but the process checks the increase in margin of error for each increase in muscle fiber activation for that muscle. The invention uses equations like the industry standard Force-Velocity model. It states that as the velocity of the muscle contraction increases, the force output decreases. Typically this equation is

F=F₀b−av/b+v Equation 1

Where F is the instantaneous force, F₀ is the force produced in isometric contraction, v is the current contraction speed, and a and b are constants.

Similarly to the SEMG 201, the electrocardiogram 201, or EKG 201, measures the electromagnetic activity. Instead of measuring the electromagnetic activity of a muscle, the EKG 201 or ECG 201 measures the electromagnetic activity of the heart 300. This data is used primarily for monitoring the heart rate of the user, original user and/or end user. Other analytics include the estimated respiration rate and monitor abnormalities. These abnormalities include heart attacks, a murmur, seizures, cardiac dysrhythmias, fainting, and other abnormalities. Even though the process searches for key signs in the abnormalities, the main purpose to monitor the heart rate through the ergonomic or athletic motion 300.

Common problems include baseline wander, power line interference, and noise correction. Baseline wander is a low frequency noise that has non-linear or non-stationary tendencies. Baseline wander can be solved by a cut off frequency at 0.05 Hz, using a capacitor, or a high pass filter in the software. Power line interference is caused by electromagnetic fields (EMF), electromagnetic interference by a power line, alternating current fields, improper grounding, or things like air conditioners. It can be fixed by a notch filter at 50/60 Hz in the software. Nosie correction can use a flexible digital filter block to fix it.

Some other filters to consider, but not limit the scope to, are infinite impulse response filters, finite impulse response filters, adaptive filters, or a wavelet transform filter. A high pass filter removes low frequency signals. A low pass filter removes high frequency signals. An example is a Gaussian impulse response, which is a time varying low pass filter with variable frequency.

The EKG 201 may or may not use signal recognition algorithms in this invention. The algorithm collects the filtered data and separates the points, segments, intervals, waves, and complex into separate matrices. The algorithm runs statistical analysis on the individual matrices to compare current results with past results and to find statistical trends in the signal. Some examples include the amount of duration of intervals, amplitude of waves, beats per minute, and interval voltage. The statistical analysis metrics are then compared to the norm with the individual and common warning signs. A common warning sign is if the P wave amplitude exceeds 3 mm or 0.3 mV. If it does exceed those nominal values it represents right atrial enlargement. Any abnormalities, with respect to the activity levels (calculated from the IMUs 202 and SEMG 201), will immediately be displayed on the preferred device 205. The signal recognition algorithm can also be compared to the activity and will provide insights of the performance.

As formally introduced by the simulated body segments, the motion recognition algorithm 425 uses a model or a series of algorithms to decipher what motion 300 is being performed. For example, the algorithm is able to distinguish between a baseball throw, a golf swing, and a runner. The motion recognition algorithm 425 is used continuously throughout the duration of the user, original user or end user, wearing the garment. An example includes recognizing a real time motion 300 from the end user to recognize when an attempt to reproduce the original user's motion and count the number of recognized motions attempts, or repetitions. As stated before, gait, or motion 300, recognition, may use models including, but not limited to, Convolutional Neural Network (or CNN), Deep Convolutional Neural Network (DCNN), Long-Short-Term memory (or LSTM), generalized recurrent units (or GRU), artificial neural network (or ANN), Bayesian Probability, genetic algorithms, Support Vector Machines, Naïve Bayes, Multi-Layer Perceptron, Random Forrest or any combination or such.

In a simple model the motion recognition algorithm 425 uses multiple motions 300 as a framework for the algorithm. Meaning a user will complete and label different golf swings 300. The IMU 202 values of the golf swings 300 are labeled, archived 450, and a large standard deviation is applied to those values. This builds the frame work for an archived motion 300 recognition process, or range of motions 300 considered a golf swing 300. The IMU 202 ranges, or IMU 202 values with the large standard deviation applied, are used to track when a motion 300 occurs. More specifically, the IMU 202 ranges, with respect to time t, create a data set, or parameters, to test every motion 300 against. For example, a golf swing 300 has key attributes each IMU 202 will follow. The golf swing 300 will deviate throughout the round and will completely change over time, but the overall key attributes of the golfers swing 300 will always be recognizable through the IMU 202 ranges. When a motion 300 is recognized, the archived data will be labeled for future data retrieval. Using the motion 300 recognition and analytics 600 throughout the motion 300, this invention claim's the ability to count the number of repetitions in a set and recognize an attempt at an archived motion. The invention continuously updates the repeated motion's data 300 and develops analytics 600 on the motion 300 to inform and improve the user's ergonomic and athletic motion 300.

The invention may use a visual approach 900 to demonstrate, or display, the motion. The visual approach uses kinematics of rigid body to show the motion 300 and analytics through a 3D avatar 900. Each body parts motion is displayed 900 via the exact IMU 202 values at its respective time. By inserting each IMUs 202 values in the avatars 900 exact location with respect to time t, the avatar 900 produces an exact digital replica of motion. The fiber activation rates are then added to the avatar 900 with respect to time t. This shows the amount of muscle fiber activation for each muscle throughout the motion. It is displayed 900 through the avatars 900 muscles, which are located in the respective locations, and gets darker when more muscle fiber activation occurs. As to say, the lighter colored muscles need less muscle fiber activation than the darker colored muscles.

The invention may also provide biofeedback after each attempt the end user completes. Biofeedback between the original user's motion and end user's motion is displayed 900 in three variations. If any deviations are present between the two motions, the biofeedback algorithm calculates the exact amount of deviation and correlation between deviations of multiple body parts. For example, the biofeedback algorithm quantifies how much deviation the right arm experienced which was directly correlated to the deviations in the right quad. So to say, the deviations of the right quad was solely responsible for such quantified deviation value of the right arm. If the right quad was directly consistent with the original users' motion, the right quad would be normalized and the correlated deviations would not have occurred.

One variation of the biofeedback display 900 is through a qualitative display 900. The qualitative display 900 gives insight pertaining to the comparison of original user's motion and end user's motion. After the end user's motion, the qualitative display 900 will choose the body part with the largest amount of deviation from the original user's motion and give instructions like “Right quad was too far to the right at impact”. The graphical interface 900 allows the user to adjust given the qualitative instructions.

The second variation is through a vocal announcement 900. The vocal announcement 900, will calculate the end users body part with the largest amount of deviation from the original user's motion. This variation 900 will announce instructions through the preferred device's 205 speaker (e.g. “Right quad was too far to the right at impact”).

The third variation is the visual comparison biofeedback. It will transparently overlay the original user's motion and the end user's motion. This shows not only the most significant deviation from the original user's motion, but every other deviation from the end user's motion. It allows for more advanced adjustments and demonstrations. This method collects the data 300 from the sensors 200 and displays 900 them through a kinematic of rigid body avatar 900, similar to the original user's motion display 900 described above. The difference between the original user's motion display and end user's motion display is the collection of the data. Instead of using data from network systems archive storage, the data displayed is from the most recent recognized motion 300.

The invention claims using archived data 450 for the purpose of coaching another individual through an avatar 900. This process allows one user to complete a motion 300, a trick, an athletic motion, an ergonomic motion, a training regimen, or such, and archive the data 450. The original user may complete any motion while wearing the garment and archive that motion to the network system. That data 450 may be then displayed 900 on another users preferred method 204 to be recreated or attempted. The final end user can attempt to recreate the motion 300 and one of three biofeedback variations will display 900 inconsistency 600 from the initial user and the end users motion.

The process begins by a user completing a motion or a series of motions while wearing the garment. The garments sensors collects the raw data pertaining to the motion, athletic motion, ergonomic motion, or workout and analyzes the raw data. The analyzed data is turned into metrics including, but not limited to, the muscle fiber activation rates, the angular velocity of each body segment, the velocity of each body segment, the path of each body segment, and the heart rate.

The analyzed data, values, and metrics are stored and archived into the network system. The network system may collect and archive the data through a few methods. The first method sends the data from the microcontroller unit to a preferred device and then to a server. The second method sends the data from the microcontroller unit directly to the server. Complete algorithms, or portions of algorithms, can be instructed from the processor at any part of this process. The process may or may not instruct the data to be analyzed on the MCU before sending it to the preferred device. The preferred device may or may not be instructed to complete algorithms or portions of algorithms to run further analyzation of the data. The server may then be instructed to complete algorithms or portions of algorithms to run analyzation of the data.

The invention uses the motion recognition software to sort the motions into sets. An example of a set is all the repetitions in an individual workout. An individual workout is defined as a singular motion, which may be repeated to create a set. The original user may complete 10 repetitions of a bench press 3 times. The repetitions of 10 are stored together and labeled. This process of recognizing a motion, calculating the number of repetitions are repeated until the training regimen is over, or until the original user ends the training regimen process via the preferred device. Each motion, number of repetitions, and number of sets is stored in order of the original user's completion of said motions.

The original user may then edit the training regimen to their desires. These edits may include, but is not limited to, labeling the motion, editing any motion, replicating any motion, inputting the number of repetitions or sets, providing alternatives if machine is unavailable, removing non workout motions from the training regimen, or anything similar in nature. This allows the original user the capabilities to create the exact training regimen, or such, without having to complete the entire training regimen when an error occurs.

The original user may create content at any location and make it readily available through the network. The process gives the capabilities to end users to download the training regimen through the network. The download process is simply a list of all motions or training regimens displayed on the preferred device of the end user and once clicked the process downloads it to the end user's preferred device.

The downloaded data is displayed on the end users device through an avatar. The data from the original user is combined with kinematics to produce a motion through the avatar. The muscle fiber activation rates may be displayed through a color code in each respective muscles locations. The color code makes the muscles turn red when a high percentage of activation occurs and turns blue when the activation levels are minimal.

The process may or may not give written instructions or a visual display as an overview. This overview quickly informs the end user what exactly the motion or training regimen entails. This may be a list of each individual workout with the number of repetitions in each set and the order of each individual workout. This may also be a specific motion used to help with a hitch in a golf swing. The overview informs the end user all the details of the motion or training regimen.

Once the end user is prepared to begin the downloaded motion or training regimen, the first step in the training regimen is displayed. The first step may be the first individual workout or the first motion to be completed in the training regimen. The motion is displayed through the avatar to show proper form and to instruct the athlete how to complete the motion.

The motion recognition software is continuously scanning the data for the completion or attempt of the motion. Once the motion recognition software confirms the motions attempt, the attempt is displayed in various different methods. One method displays the actual motion through an overlay on top of the desired motion, or motion displayed through the training regimen data. This allows the end user to visually see where deviations are occurring. Another method verbally states deviations between the downloaded motion and the real time motion. The last method informs the athlete of the deviations through qualitative display. An example of the written form includes “Straighten your back during your next repetition”. This process continues for each repetition and set until the training regimen is completed.

In a variation of the process, the number of sets and individual workouts include using metrics as key indicators for the amount of repetitions or the exact individual workouts. The number of repetitions or series of individual workouts can change depending on fatigue levels, heart rate, time, deviations, and others. For example, the training regimen may begin with completing as many sit-ups in 1 minute as possible and depending on fatigue levels, the athlete may either be instructed to complete as many push-ups or knee push-ups as possible in the next minute. Other changes may include changing from one individual workout to another, changing the amount of weights, the difficulty of the motion, or others similar. This feature is completed before the original user sends the motion, training regimen, or such to the network systems database. The original user can make edits or changes to the training regimen, or such, as described above. This feature allows the original user to fork the steps of the specific training regimen based on the end user's physiological metrics, amount of deviations, failure or success of an individual workout (or step in the training regimen), or such. The original user may choose a specific time period (actual time or number of steps in the training regimen), specific motion, or such for the invention to monitor for specific physiological metrics, amount of deviations, failure or success of an individual workout, or such. Once the original user chooses the time period and the values and metrics to monitor for, the user can choose either to change the complete training regimen, the specific time period, or change the training regimen until the key values and metrics return to normal. This invention claims customized training regimens from a coach, or original user, which can differentiate based on key indicators collected from the sensors on the garment.

Once any data 300 is collected, the data is stored 400 and archived 450. This data includes all biometric sensors 200 in use during every motion. It also includes any relevant analytics through any of the inventions processes.

Information collected and analyzed through the invention's process may be archived 450. The archival storage system 450 may be in contact with the preferred device 204 or the data may be archived 450 on the preferred device 204.

In various embodiments may include multiple users creating content for training regimen, or such, on multiple devices. Multiple devices can adjust any feature, label any motion, or anything such in nature.

In various embodiments, the network system may include multi directional communication and data transfer capabilities. This allows users to have real time displaying capabilities and real time communication between the users.

In other variations of the biometric system 200, respiration sensors, galvanic skin response sensors, temperature sensors, global positioning system sensors, vibration sensors, bio impedance sensors, bend-angle measurement sensors, light detection and ranging (LIDAR), and any other sensors relevant for the data collection process. Another variation of the biometric system 200 includes added any of the previous listed sensors 200 to a user's equipment coupled with the garment 100.

In other variations of the biometric system 200, added or removed sensors 200 are included on the user. This includes more or less SEMG 201, IMU 202, EKG 201 sensors 200 then what is described in FIG. 4, FIG. 5, FIG. 6, FIG. 7, FIG. 8, and FIG. 9. The exact amount of sensors 200 or their location attached to the clothing 100 is not limited in this patent, but the ability to use the sensors 200, specifically IMU 202 and electrodes 201 (more specifically SEMG and EKG), attached to the clothing 100 or garment 100 to collect a motions data, archive the motion, and display the archived motion on an end user's preferred device. This concept also has the capabilities for live training sessions. In other variations, the invention can include a short sleeve compression shirt instead of a long sleeve compression shirt.

In other variations of the biometric system 200, added sensors not attached to the clothing 100 are stored with archived motion. For example, a glove's data not attached to the garment 100 that has sensors and has capabilities of connecting to the network can archived with training regimen.

Another variation of the IMU sensors 202 calibration uses key motions. This variation uses the said motion key to calibrate the IMUs 202 when attached to the garment 100. This motion key is customizable to the user's preference, but a suggested motion key is given. The motion key's motion 300, when recognized or instructed, is compared to the Kalman Filter's results. If the delta between the two are significant, the appropriate data in the Kalman filter or such is calibrate. For example, by touching the IMUs 202 on the right arm and chest in a sequence, the left arm's IMUs 202 will calibrate for any deviations at all. The SEMG 201 values 201 of the left arm will confirm the exact time when the user touches the location for the calibration process through abnormalities in the SEMG data.

The present invention is well adapted to carry out the objectives and attain both the ends and the advantages mentioned, as well as other benefits inherent therein. While the present invention has been depicted, described, and is defined by reference to particular embodiments of the invention, such reference does not imply a limitation to the invention, and no such limitation is to be inferred. The depicted and described embodiments of the invention are exemplary only, and are not exhaustive of the scope of the invention. Consequently, the present invention is intended to be limited only be the spirit and scope of the claims, giving full cognizance to equivalents in all respects.

I claim:

1. A method for providing live and archived athletic and ergonomic motions lessons to remote users comprising:
identifying, by one or more computing devices and one or more sensors, a first user's motion, the motion having metrics and analytics;
providing information about coaching lesson that can be accessed via a digital communication network to a second user's;
sending coaching lesson content from a server to a second user's device;
displaying said coaching lesson on second user's device;
detecting, by one or more computing devices and one or more sensors, a second user's deviations from the coaching lesson content;

2. The method of claim 1, wherein the content sent to the second user's device is archived content provided from a database.

3. The method of claim 1, wherein the content sent to the second user's device is streamed in real time.

4. The method of claim 1, further comprising displaying content comprising the selected coaching lesson on the display screen associated with the second user.

5. The method of claim 1, wherein the detected deviations from the second user are sent to the first user's device is archived content provided from a database.

6. The method of claim 1, wherein the detected deviations are sent to the first user's device is streamed in real time.

7. A computer-implemented coaching method comprising:
identifying, by one or more computing devices and one or more sensors, a first user's motion, the motion having metrics and analytics;
providing information about coaching lesson that can be accessed via a digital communication network to a second user's;
sending coaching lesson content from a server to a second user's device;
displaying said coaching lesson on second user's device;
detecting, by one or more computing devices and one or more sensors, a second user's deviations from the coaching lesson content;

8. The method of claim 7, wherein the content sent to the second user's device is archived content provided from a database.

9. The method of claim 7, wherein the content sent to the second user's device is streamed in real time.

10. The method of claim 7, further comprising displaying content comprising the selected coaching lesson on the display screen associated with the second user.

11. The method of claim 7, wherein the detected deviations from the second user are sent to the first user's device is archived content provided from a database.

12. The method of claim 7, wherein the detected deviations are sent to the first user's device is streamed in real time.

13. A system comprising one or more computing devices and one or more sensors configured to:
identifying, by one or more computing devices and one or more sensors, a first user's motion, the motion having metrics and analytics;
providing information about coaching lesson that can be accessed via a digital communication network to a second user's;
sending coaching lesson content from a server to a second user's device;
displaying said coaching lesson on second user's device;
detecting, by one or more computing devices and one or more sensors, a second user's deviations from the coaching lesson content;

14. The method of claim 13, wherein the content sent to the second user's device is archived content provided from a database.

15. The method of claim 13, wherein the content sent to the second user's device is streamed in real time.

16. The method of claim 13, further comprising displaying content comprising the selected coaching lesson on the display screen associated with the second user.

17. The method of claim 13, wherein the detected deviations from the second user are sent to the first user's device is archived content provided from a database.

18. The method of claim 13, wherein the detected deviations are sent to the first user's device is streamed in real time.

19. A non-transitory computer-readable medium on which instructions are stored, the instructions, when executed by one or more processors cause the one or more processors to perform a method, the method comprising:
identifying, by one or more computing devices and one or more sensors, a first user's motion, the motion having metrics and analytics;
providing information about coaching lesson that can be accessed via a digital communication network to a second user's;
sending coaching lesson content from a server to a second user's device;
displaying said coaching lesson on second user's device;
detecting, by one or more computing devices and one or more sensors, a second user's deviations from the coaching lesson content;

20. The method of claim 19, wherein the content sent to the second user's device is archived content provided from a database.

21. The method of claim 19, wherein the content sent to the second user's device is streamed in real time.

22. The method of claim 19, further comprising displaying content comprising the selected coaching lesson on the display screen associated with the second user.

23. The method of claim 19, wherein the detected deviations from the second user are sent to the first user's device is archived content provided from a database.

24. The method of claim 19, wherein the detected deviations are sent to the first user's device is streamed in real time.
